(19) United States
(12) Patent Application Publication
(10) Pub. No.: US 2017/0362662 A1
(43) Pub. Date: Dec. 21, 2017

(54) NOVEL RNA-BIOMARKER SIGNATURE FOR DIAGNOSIS OF PROSTATE CANCER

(71) Applicant: FRAUNHOFER-GESELLSCHAFT ZUR FÖRDERUNG DER ANGEWANDTEN FORSCHUNG E.V., München (DE)

(72) Inventors: Friedemann Horn, Leipzig (DE); Jörg Hackermüller, Leipzig (DE); Sabina Christ, Leipzig (DE); Kristin Reiche, Leipzig (DE); Manfred Wirth, Dresden (DE); Michael Fröhner, Dresden (DE); Susanne Füssel, Dresden (DE)

(73) Assignee: FRAUNHOFER-GESELLSCHAFT ZUR FÖRDERUNG DER ANGEWANDTEN FORSCHUNG E.V., München (DE)

(21) Appl. No.: 15/532,381

(22) PCT Filed: Dec. 1, 2015
(86) PCT No.: PCT/EP2015/078183

§ 371 Date: Jun. 1, 2017

(30) Foreign Application Priority Data
Dec. 1, 2014 (EP) 14195709.2

(51) Int. Cl. C12Q 1/68

(52) U.S. Cl. CPC C12Q 1/6886; C12Q 2600/158

(57) ABSTRACT

The invention relates to the identification and selection of differentially expressed transcripts (biomarker) in tumour cells. Specific determination of the level of these biomarkers can be used for screening and diagnosis of prostate cancer. Clinical application of assays based on these biomarker help reduce the high number of false positives of current standard screening assays.

### FIELD OF THE INVENTION

The present invention is in the field of biology and chemistry. In particular, the invention is in the field of molecular biology. More particular, the invention relates to the analysis of RNA transcripts. Most particularly, the invention is in the field of diagnosing prostate cancer.

### BACKGROUND

Prostate cancer is the most frequently diagnosed cancer in men. In 2012, the annual number of newly diagnosed prostate cancer cases was reported as approximately 240,000 cases in the United States and approximately 360,000 in the European Union, 68,000 of which in Germany. In the United States, lifetime risks for prostate cancer diagnosis and for dying of prostate cancer are currently estimated at 15.9% and 2.8%, respectively. Despite widespread screening for prostate cancer and major advances in the treatment of metastatic disease, prostate cancer remains the second most common cause of cancer death for men with over 250,000 deaths each year in the Western world.

Currently, testing of prostate-specific antigen (PSA) serum levels and the digital rectal examination represent the two major screening methods. Patients showing abnormal results usually are advised to have a prostate biopsy performed. This has however significant consequences. The lack of specificity of PSA screening which produces high numbers of false positives results in unnecessary prostate biopsies performed annually on millions of men worldwide (overdiagnosis). In addition, taking biopsies carries a substantial risk for infectious complications. Therefore, there is an urgent need for a more sensitive and specific diagnostic assay for early prostate cancer diagnosis to improve prostate cancer screening and to avoid the high numbers of unnecessarily taken prostate biopsies. The present invention addresses this problem by providing a set of biomarkers for the screening and diagnosis of prostate cancer.

### SUMMARY OF THE INVENTION

Transcripts differentially expressed in tumour and control tissues were identified by Next Generation Sequencing of 64 samples of prostate cancer patients and controls and validated by microarray and qRT-PCR analyses of 256 and 56 samples, respectively. The invention describes RNA biomarkers, which had not so far been found to be suitable for use in the diagnosis of prostate cancer.

The invention relates to a method for the diagnosis of prostate cancer comprising the steps of analysing the expression levels of at least two nucleic acids in a sample of a patient, wherein said at least two nucleic acids are selected from at least two groups of nucleic acids, wherein said groups consist of:
Group 1: SEQ ID NO 1
Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38
   wherein, if the expression levels of said at least two nucleic acids are above a threshold value, the sample is designated as prostate cancer positive.

The invention also relates to a set of nucleic acids that hybridize under stringent conditions to the nucleic acids in the groups above.

The invention further relates to the use of the method for the diagnosis of prostate cancer.

The invention also relates to a kit for the diagnosis of prostate cancer comprising at least two primers or probes, which hybridize under stringent conditions to at least two nucleic acids, wherein said at least two nucleic acids are selected from at least two groups of nucleic acids, wherein said groups consist of:
Group 1: SEQ ID NO 1
Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38

### Definitions

The following definitions are provided for specific terms, which are used in the application text.

As used herein, “nucleic acid(s)” or “nucleic acid molecule” generally refers to any ribonucleic acid or deoxyribonucleic acid, which may be unmodified or modified DNA. “Nucleic acids” include, without limitation, single- and double-stranded nucleic acids. As used herein, the term “nucleic acid(s)” also includes DNA as described above that contain one or more modified bases. Thus, DNA with backbones modified for stability or for other reasons are “nucleic acids”. The term “nucleic acids” as it is used herein encompasses such chemically, enzymatically or metabolically modified forms of nucleic acids, as well as the chemical forms of DNA characteristic of viruses and cells, including for example, simple and complex cells.

The terms “level” or “expression level” in the context of the present invention relate to the level at which a biomarker is present in a sample of a patient. The expression level of a biomarker is generally measured by comparing its expression level to the expression level of one or several housekeeping genes in a sample for normalisation. The sample from the patient is designated as prostate cancer positive if the expression level of the biomarker exceeds the expression level of the same biomarker in an appropriate control (for example a healthy tissue) by a set threshold value.

The term, “analysing a sample for the presence and/or level of nucleic acids” or “specifically estimate levels of nucleic acids”, as used herein, relates to the means and methods useful for assessing and quantifying the levels of nucleic acids. One useful method is for instance quantitative reverse transcription PCR. Likewise, the level of RNA can also be analysed for example by northern blot, next generation sequencing or after amplification by using spectrometric techniques that include measuring the absorbance at 260 and 280 nm.

As used herein, the term “amplified”, when applied to a nucleic acid sequence, refers to a process whereby one or more copies of a particular nucleic acid sequence is generated from a nucleic acid template sequence, preferably by the method of polymerase chain reaction. Other methods of amplification include, but are not limited to, ligase chain reaction (LCR), polynucleotide-specific based amplification (NSBA), or any other method known in the art.

The term “correlating”, as used herein in reference to the use of diagnostic and prognostic marker(s), refers to comparing the presence or amount of the marker(s) in a sample from a patient to its presence or expression level in a sample from a person known to suffer from, or is at risk of suffering from, a given condition. A marker expression level in a patient sample can be compared to a level known to be associated with a specific diagnosis.

As used herein, the term “diagnosis” refers to the identification of the disease, in this case prostate cancer, at any stage of its development, and also includes the determination of predisposition of a subject to develop the disease.

The term “Ensembl gene ID ENSG00000245750.3” relates to a gene ID sequence annotation by Ensembl. Transcripts that belong to the same gene ID may differ in splice events, exons, and can give rise to very different proteins. These are isoforms, arising from alternative splicing. The Ensembi gene ID has several equivalents in other annotation systems such as for example RP11-279F6.1, or locus (hg19) Chr15: 69,755,365-69,863,775 (+). Any equivalent to this Ensembl annotation can be used in its place.

The term “Ensembl gene ID ENSG00000255545.3” relates to a gene ID sequence annotation by Ensembl. Transcripts that belong to the same gene ID may differ in splice events, exons, and can give rise to very different proteins. These are isoforms, arising from alternative splicing. The Ensembl gene ID has several equivalents in other annotation systems such as for example RP11-627G23.1, or locus (hg19) Chr11: 134,306,367-134,375,555 (+). Any equivalent to this Ensembl annotation can be used in its place.

As used herein, the term “fluorescent dye” refers to any chemical that absorbs light energy of a specific wavelength and re-emits light at a different wavelength. Fluorescent dyes suitable for labelling nucleic acids include for example FAM (5-or 6-carboxyfluorescein), VIC, NED, Fluorescein, FITC, IRD-700/800, CY3, CY5, CY3.5, CY5.5, HEX, TET, TAMRA, JOE, ROX, BODIPY TMR, Oregon Green, Rhodamine Green, Rhodamine Red, Texas Red, Yakima Yellow, Alexa Fluor, PET and the like.

As used herein, “isolated” when used in reference to a nucleic acid means that a naturally occurring sequence has been removed from its normal cellular (e.g. chromosomal) environment or is synthesised in a non-natural environment (e.g. artificially synthesised). Thus, an “isolated” sequence may be in a cell-free solution or placed in a different cellular environment.

As used herein, a “kit” is a packaged combination optionally including instructions for use of the combination and/or other reactions and components for such use. If the kit contains nucleic acids, the kit may also comprise synthetic or non-natural variants of said nucleic acids. A synthetic or non-natural nucleic acid is to be understood as a nucleic acid comprising any chemical, biochemical or biological modification, such that the nucleic acid does not appear in nature in this form. Such modifications include, but are not limited to, labelling with a fluorescent dye or a quencher moiety, a biotin tag, as well as modification(s) in the backbone of a nucleic acid, or any other modification that distinguishes the element from its natural counterpart. The same applies also to other natural compounds such as proteins, lipids and the like.

The term “patient” as used herein refers to a living human or non-human organism that is receiving medical care or that should receive medical care due to a disease, or is suspected of having a disease. This includes persons with no defined illness who are being investigated for signs of pathology. Thus the methods and assays described herein are applicable to both, human and veterinary disease.

The term “primer” as used herein, refers to an nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. Preferably, primers have a length of from about 15-100 bases, more preferably about 20-50, most preferably about 20-40 bases. The factors involved in determining the appropriate length of primer are readily known to one of ordinary skill in the art. Optionally, the primer can be a synthetic element, in the sense that it comprises a chemical, biochemical or biological modification. Such modifications include, but are not limited to, labelling with a fluorescent dye or a quencher moiety, or a modification in the backbone of a nucleic acid, or any other modification that distinguishes the primer from its natural nucleic acid counterpart.

The term “probe” refers to any element that can be used to specifically detect a biological entity, such as a nucleic acid, a protein or a lipid. Besides the portion of the probe that allows it to specifically bind to the biological entity, the probe also comprises at least one modification that allows its detection in an assay. Such modifications include, but are not limited to labels such as for fluorescent dyes, a specifically introduced radioactive element, or a biotin tag. The probe can also comprise a modification in its structure, such as a locked nucleic acid.

The term “sample” as used herein refers to a sample of bodily fluid or tissue obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analysed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples as well as circulating tumour cells in blood or lymph, any tissue suspected to contain metastases as well as any source that may contain prostate tumour cells or parts thereof, including vesicles like exosomes, microvesicles, and others as well as free or protein-bound RNA molecules derived from prostate tumour cells. Preferably, the sample is a blood sample, most preferably a serum sample or a plasma sample. Importantly, urine (particularly after digital rectal examination) and ejaculate belong to the most preferable samples. Tissue samples may also be biopsy material or tissue samples obtained during surgery.

The term “area under the curve (AUC)” as used herein describes the area under the curve of a receiver operating characteristic (ROC) or ROC curve. The AUC relates to how specific and sensitive a biomarker is. A perfect marker (AUC=1.0) would yield a point in the upper left corner or coordinate (0,1) of the ROC space, representing 100% sensitivity (no false negatives) and 100% specificity (no false positives).

The term “p-value” relate to the probability of obtaining the observed sample results (or a more extreme result) when the null hypothesis is actually true, i.e. there are no differences between means for groups. The smaller the p-value, the higher the likelihood that the alternative hypothesis explains the observed results better than the null hypothesis.

The term “adjusted p-value” refers to p-values which have been adjusted for multiple comparisons (number of genes/probes tested). The method applied is detailed in the experimental section.

### DETAILED DESCRIPTION OF THE INVENTION

The invention describes a method of diagnosis of prostate cancer. This method comprises analysing a sample taken from a patient and specifically determine the level of a combination of biomarkers in said patient sample. The result for each biomarker is then correlated to a threshold value and in the case it is above that threshold value, said patient sample is designated as prostate cancer positive.

The invention relates to several groups of sequences comprising SEQ ID NOs 1 to 42. The sequences are listed below. Due to space constraints, only the first 100 nucleotides are listed. The remaining part of the sequence can be found in the sequence protocol.

In particular the invention relates to 8 distinct groups of sequences:
Group 1: SEQ ID NO 1
Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38

The nucleic acid sequences of SEQ ID NO 3 and 11 correspond to spliced transcripts from the locus (hg19): Chr2: 1,550,437-1,629,191 (−).

The transcripts SEQ ID NO: 36 and 38 are respectively 38 342 nucleotides and 346 832 nucleotides in length. The inventors surprisingly found that these two nucleic acids correspond to single long transcripts. The detection of the expression level of any part of these long transcripts is therefore suited for the method of the present invention.

There are two types of sequences. First, some transcripts are known sequences that are already annotated in relevant databases. They are identified by their respective annotations. Second, new transcripts were identified that are not yet annotated. They are designated here as follows: XLOC_ followed by a number. These designations provide information about the genomic origins of the transcripts, but may not necessarily represent the whole sequence of a given transcript. The sequences as detected may in some cases be longer or shorter. In the case of XLOC transcripts, if fragments are detected, these fragments may be as small as 1000, 500, 400, 300, 200, 150, 100, 50, 40, 30, 20, 10, 9, 8, 7, 6 or 5 nucleotides.

TABLE 1
List of SEQ ID NOs. SEQ ID NOs 1 to 42 are listed together with the
corresponding transcript and gene annotations. The first 100
nucleotides of each SEQ ID NO are shown.

| | Trans- | | |
|---|---|---|---|
| SEQ ID | cript | Gene/transcript annotation | Sequence |
| 1 | 1 | Retro-RPL7 | Ttttccggctggaacca |
| | | Ensemble-ID gene: ENSG00000242899.1 | tggagggtgttgaagag |
| | | Locus (hg19): Chr3: 131,962,301- | aagaagaaggttcctgc |
| | | 131,963,125 (−) | tgtgccagaaaccctta |
| | | Ensemble-ID transcript(s): | agaaaaagtgaaggaat |
| | | ENST00000479738.1 | ttcacagagctgaag |
| 2 | 2 | XLOC_133897 | gcccgcttctgtgactc |
| | | Ensemble-ID gene: none | caccccttacggaaagt |
| | | Locus (hg19): chr20: 45,377,600- | ctatgggactctctgaa |
| | | 45,380,719 (−) | atgtatgagtgatactg |
| | | Ensemble-ID transcript(s): none | ttagaaagcggcaagaa |
| | | Includes GenBank entries: | aatgaaaagaaaacg |
| | | AK128800.1, BC065739.1 | |
| 3 | 3 | AC144450.2 | attgcccacagccggat |
| | | Ensemble-ID gene: ENSG00000203635.2 | ccacggtgactaatctc |
| | | Locus (hg19): Chr2: 1,624,282- | cgggaaggcgtccagcg |
| | | 1,629,191 (−) | tgagccgtgaggcctgc |
| | | Ensemble-ID transcript(s): | acctgcgccggacttca |
| | | ENST00000366424 | ccactcaccaggagt |
| 4 | 4 | RP11-279F6.1 | caggaatgggctggggc |
| | | Ensemble-ID gene: ENSG00000245750.3 | gcgtttgtagttgggaa |
| | | Locus (hg19): Chr15: 69,755,365- | tcctgagcccgggctgt |
| | | 69,863,775 (+) | tgcttggaggactcggg |
| | | Ensemble ID trancript: | agcagcagtggatttcg |
| | | ENST00000558633 | gcgttaccaggagag |
| 5 | 4 | ENST00000558309 | ttcggcgttaccaggag |
| | | | agctatgtataggaatg |
| | | | ccgctatggaaagacat |
| | | | ccaggacaccttgttaa |
| | | | gtgaaaaaagacatgcc |
| | | | accattagggcttca |
| 6 | 4 | ENST00000560882 | gaggcccgacattgtgc |
| | | | tggggaaggagctccag |
| | | | aaagggccatcctttct |
| | | | gttttggttcagtatct |
| | | | gaacacttttgctaaag |
| | | | gtctctggaaagctc |
| 7 | 4 | ENST00000559029 | Gactggagaggccagca |
| | | | cgcacagtgacttaatc |
| | | | caagaagatggaataaA |
| | | | aaggcctacctcattgg |
| | | | gctcgtgtgggtgagga |
| | | | gaactgaagagtctg |
| 8 | 4 | ENST00000558781 | Ctgggcttccagcttcc |
| | | | aagccttctacctgtgg |
| | | | aatgcttggtccaatgT |
| | | | ctggggcacccactctt |
| | | | actccaaactcctccag |
| | | | atctgcagagtggcc |
| 9 | 4 | ENST00000498938 | Ggagctggttccaggaa |
| | | | agaagggcacatgagca |
| | | | aacatgatggccccttt |
| | | | atgagaggtaatttact |
| | | | gaaatgcacagcgatta |
| | | | cctgctcacccagcc |
| 10 | 4 | ENST00000559477 | aggaacttggaataact |
| | | | tgcagtgtcttgcagta |
| | | | ttgtgaaaccagcaacT |
| | | | tgttcacaattcttctg |
| | | | aatttcttgggaaattt |
| | | | gaagtggagtacctg |
| 11 | 5 | AC144450.1 | cagttttcacaggcctg |
| | | Ensemble-ID gene: ENSG00000228613.1 | tgtgccgagagtgttcc |
| | | Locus (hg19): Chr2: 1,550,437- | ttaccattttttcatta |
| | | 1,623,885 (-) | ttattctgctaaggagg |
| | | Ensemble-ID transcript (s): | atttttagacattatgt |
| | | ENST00000438247.1 | tcctagtcaagccct |
| 12 | 6 | AC012531.25 | caagacagaggcaagca |
| | | Ensemble-ID gene: ENSG00000260597.1 | gagaaggcatagcagca |
| | | Locus (hg19): Chr12: 54,413,694- | gogaccggcgctctgtt |
| | | 54,416,373 (+) | ttcattttccactctgg |
| | | Ensemble-ID transcript (s): | ccaggggataaactgga |
| | | ENST00000562848.1 | ccccagtggactcca |
| 13 | 7 | XLOC_068574 | ggtaacatgaaaataat |
| | | Ensemble-ID gene: none | ggatgagcagttcaact |
| | | Locus (hg19): chr14: 62,653,302- | atattaaaaataaacgt |
| | | 62,655,723 (+) | ggttaagagtgctcacc |
| | | Ensemble-ID transcript(s): none | ttaagtgtaggatttga |
| | | | aagtgtaggctctaa |
| 14 | 8 | RP1-207H1.3 | Tgaagcccatgagccac |
| | | Ensemble-ID gene: ENSG00000231150.1 | tagaagccacatgttct |
| | | Locus (hg19): chr6: 38,890,805- | gccatgtggagaagaat |
| | | 38,920,875 (−) | gagagagtacatcctca |
| | | Ensemble ID transcript: | aattgaggtgtggcatg |
| | | ENST00000416948.1 | atgatttggctgccc |
| 15 | 8 | ENST00000453417.1 | ctttcaagggcctgtgc |
| | | | ctgtggtaactgtctat |
| | | | gagccaggtatatctga |
| | | | agcatatttgacaacag |
| | | | aaaaagttaatgtaatt |
| | | | ttcaaaggaaaaacg |
| 16 | 8 | ENST00000418399.1 | atatctgaagcatattt |
| | | | gacaacagaaaaagtta |
| | | | atgtaattttcaaagga |
| | | | aaaacgccaactttttt |
| | | | caaaaaggaaacagcaa |
| | | | ctggagagcagattt |
| 17 | 9 | XLOC_016724 | atcccctctgagaattt |
| | | Ensemble-ID gene: none | atcagaaaaacaagcaa |
| | | Locus (hg19): chr1: 177,827,793- | taagtgagaccaacgtt |
| | | 177,841,757 (−) | gtgaggtattaactcgg |
| | | | aaccgtcatctatcctt |
| | | | gtggagaaaaacccg |
| 18 | 10 | RP11-314O13.1 | ttctttttgtttgotgc |
| | | Ensemble-ID gene: ENSG00000260896 | cttccgtagaagatgtg |
| | | Locus (hg19): Chr16: 80,862,632- | gcttgctcatgcttgac |
| | | 80,926,492 (-) | ttctgccatggttgtga |
| | | Ensemble ID transcript: | ggcctccccagccatgt |
| | | ENST00000562231 | ggaactgttttcagg |
| 19 | 10 | ENST00000569356 | Aggggtttccgcttttg |
| | | | cttcttcctcattttct |
| | | | cttgctgctgccatttT |
| | | | cgcctcccgccatgatt |
| | | | ctgaggcctccccagct |
| | | | atgtggaactgtaag |
| 20 | 10 | ENST00000561519 | Aaaagactatctcttcc |
| | | | cattgaattaaattgga |
| | | | actttggaatcttaatA |
| | | | gaaaaccaactgacttg |
| | | | gcttggttttcaggtgc |
| | | | tggttccatggctct |
| 21 | 10 | ENST00000563626 | Cttgctcatgcttgact |
| | | | tctgccatggttgtgag |
| | | | gcctccccagccatgtG |
| | | | gaactgttttcaggtgc |
| | | | tggttccatggctcttc |
| | | | ctgagccgaaaataa |
| 22 | 11 | XLOC_167596 | Ctctttctctoottctc |
| | | Ensemble-ID gene: none | ccttccttcctccctcc |
| | | Locus (hg19): chr4: 67,964,836- | ctccctctcttcctctc |
| | | 67,975,652 (−) | ttttctttctttctttc |
| | | | tctttctttctttcttt |
| | | | ctttctttctttctt |
| 23 | 12 | XLOC_167595 | aaacatacgtgtgcatg |
| | | Ensemble-ID gene: | tgtctttatggcagcat |
| | | Locus (hg19): chr4: 67,946,236- | gatttataatcctttgg |
| | | 67,964,614 (−) | ggatatactcagtaatg |
| | | | ggatggctgggtcaaat |
| | | | ggtatttctagttct |
| 24 | 13 | XLOC_156132 | agtatgtgcatttgtac |
| | | Ensemble-ID gene: none | cttgctttgttttcctc |
| | | Locus (hg19): chr3: 193,632,725- | aactttgtgcttgtttC |
| | | 193,636,178 (−) | tgtaattccctcattca |
| | | | ttcctacctctgcatgc |
| | | | ttgaaagttctttgt |
| 25 | 14 | XLOC_156120 | accaaaggacatgcgaa |
| | | Ensemble-ID gene: none | aacttttgggtgtgatg |
| | | Locus (hg19): chr3: 193,580,748- | gatatagtcataatctt |
| | | 193,608,459 (−) | tattgtggtgactgttt |
| | | | cacacatgtgtacatat |
| | | | atcacaactcatcaa |
| 26 | 15 | RP11-627G23.1 | cttcctcggggtttgct |
| | | Ensemble-ID gene: ENSG00000255545.3 | tccaggcctgactttta |
| | | Locus (hg19): Chr11: 134,306,367- | ctcccctttctaagtgt |
| | | 134,375,555 (+) | gcagatgggatgtgctt |
| | | Ensemble ID transcript: | ctccacaggaggcccca |
| | | ENST00000533390 | cggcttccccacccc |
| 27 | 15 | ENST00000531319 | ctgtctcaagcctccaa |
| | | | tcaacagatcagacagc |
| | | | ttgtactcacaggccaa |
| | | | ggacacgtggaaagagg |
| | | | ctcaattttctagatgg |
| | | | gtggcaacagccatg |
| 28 | 15 | ENST00000528482 | gaggcagccatgactgg |
| | | | ccacttcatgtgctcct |
| | | | ggagaagggcttgcacc |
| | | | agccgttttcaggaaag |
| | | | tcaagcagctgttgact |
| | | | cctgagtctgggtga |
| 29 | 15 | ENST00000532886 | caaatgcctggcagcgt |
| | | | cctcggtgcttcacctg |
| | | | ccatagccgacagtggc |
| | | | tgacctcccatgcctgt |
| | | | tgccttttctttctgtt |
| | | | ggatcagggatacac |
| 30 | 16 | XLOC_047797 | aagatgggacaattttt |
| | | Ensemble-ID gene: none | tttcctcttggtttctt |
| | | Locus (hg19): chr12: 75,378,181- | tataattattgtacccc |
| | | 75,383,176 (+) | ttttctggaataatctt |
| | | | ttcatettgttcatctg |
| | | | tcaatgcctgcttgt |
| 31 | 17 | ANKRD34B | agctgctggcccccctg |
| | | Ensemble-ID gene: ENSG00000189127.3 | ggtccagaggagccttg |
| | | Locus (hg19): Chr5: 79,852,574- | ccgccctcacctgcgca |
| | | 79,866,307 (−) | gagcctggagccgacgc |
| | | Ensemble ID transcript: | gtcacccccagcggaag |
| | | ENST00000338682 | cgcctcgctgcccgg |
| 32 | 17 | ENST00000508916 | agctcagctcagacggc |
| | | | gccctagggccgcacag |
| | | | agggtcgggcagtgccg |
| | | | gagagaggtttgaaagc |
| | | | gccgccgccaactcgac |
| | | | agcgcgtcccaggaa |
| 33 | 18 | XLOC_243739 | aaacaggaaaagaaatt |
| | | Ensemble-ID gene: none | gggatttttatgaaaaa |
| | | Locus (hg19): chr9: 79,530,077- | tgttaaaggctagctct |
| | | 79,542,427 (−) | gttaggatttcccatga |
| | | | cattgcagtggtgacat |
| | | | gqtcgtggatgtgcc |
| 34 | 19 | XLOC_198292 | tccctcccttccttcct |
| | | Ensemble-ID gene: none | tccttccttcctttctt |
| | | Locus (hg19): chr6: 148,396,831- | cccttcagtttctcttc |
| | | 148,428,362 (+) | cttctaatgccccctgt |
| | | | ccttaaaaatgtctcca |
| | | | ttcaggcactatgca |
| 35 | 20 | XLOC_068639 | ccaagatttctcatcca |
| | | Ensemble-ID gene: none | tggtttcaactaagaat |
| | | Locus (hg19): chr14: 62,931,844- | attttattctctccagt |
| | | 62,933,233 (+) | gaaattttttacaatta |
| | | | ggattgcaaaactacat |
| | | | acattcaggtagatc |
| 36 | 21 | XLOC_172083 | cactgcagtctctccct |
| | | Ensemble-ID gene: none | ccctggttcaagcaatt |
| | | Locus (hg19): chr4: 169,961,616- | ctcttgcctcagtctcc |
| | | 169,999,957 (−) | tgagtagctgggaccac |
| | | | aggcgctcaccaccacg |
| | | | catggctcatttttg |
| 37 | 22 | XLOC_172082 | agtgatccgcccgcctc |
| | | Ensemble-ID gene: none | cgcctcccaaagtgctg |
| | | Locus (hg19): chr4: 169,947,628- | ggattacaggtgtgagc |
| | | 169,961,481 (−) | cactgcgcctggccgct |
| | | | gctcttatactattttg |
| | | | aatgtaggccggccg |
| 38 | 23 | XLOC_112832 | agcagatggcatttgag |
| | | Ensemble-ID gene: none | caaacacttgcaaaagg |
| | | Locus (hg19): chr2: 123,297,707- | tgaggaagatagccatc |
| | | 123,644,538 (+) | atagctgatggaacaag |
| | | | caaaacaaaagtcataa |
| | | | ggaagaattgtactc |
| 39 | 24 | XLOC_243747 | cccgcagctgcgcccca |
| | | Ensemble-ID gene: none | cccgggccaccaagcac |
| | | Locus (hg19): chr9: 79,622,778- | ggtggagggggaacagg |
| | | 79,633,361 (−) | acactgccttcttgctt |
| | | | ctcttctctctggcatc |
| | | | tccctcttccgcccc |
| 40 | 25 | XLOC_243744 | atgtgccaccacacctg |
| | | Ensemble-ID gene: none | gctgattttttgtattt |
| | | Locus (hg19): chr9: 79,601,892- | ttagtagagatgggata |
| | | 79,606,132 (−) | tcaccatattaaccaag |
| | | | atggtctcgattacctg |
| | | | acctcgtgatccgcc |
| 41 | 26 | XLOC_126289 | cctgtgcatctaattta |
| | | Ensemble-ID gene: none | gtggggggcagacctgt |
| | | Locus (hg19): chr2: 180,988,687- | ttcacaagccaaaataa |
| | | 180,989,287 (−) | caggctgcaataactga |
| | | | ggattttatatataccc |
| | | | tgaccaaagaagttt |
| 42 | 27 | XLOC_172084 | attgtggaactgctctt |
| | | Ensemble-ID gene: none | tctccctgcgattcaga |
| | | Locus (hg19): chr4: 169,983,995- | ggggaaaagataaagcc |
| | | 169,984,246 (−) | acacagccctggggcct |
| | | | cttgcttaagaacacat |
| | | | ctcagtttaaccacc |

The biomarker PCA3 is routinely used for prostate carcinoma (PCa) diagnosis. As expected therefore, PCA3 expression levels were indicative of PCa in the subjects tested by next generation sequencing by the inventors (FIG. 2). However, it was found that the biomarker had its highest expression level in very low risk tumours (V) and decreased as the risk factor of tumours grew. This finding makes PCA3 an unreliable marker for medium- and high-risk tumours and shows the need for better prostate cancer biomarkers.

Many of the novel biomarkers found by the inventors are significantly better in terms of specificity and sensitivity than PCA3. Retro-RPL7 (SEQ ID NO: 1) for example yielded an area under the ROC curve (AUC) value of 0.935, compared to 0.851 for PCA3 (FIG. 3).

A combination of the biomarkers with SEQ ID NO:1 and SEQ ID NO: 12 yielded an AUC of 0.985 (see table 7).

The following groups of biomarkers have been found to be highly indicative in combination for prostate cancer:
Group 1: SEQ ID NO 1
Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38

A combination of biomarkers selected from all eight groups results in an AUC of 1.0 (see FIG. 6).

Hence the invention relates to a method for the diagnosis of prostate cancer comprising the steps of analysing the expression level of at least two nucleic acids in a sample of a patient, wherein said at least two nucleic acids are selected from at least two groups of nucleic acids, wherein said groups consist of:
Group 1: SEQ ID NO 1
Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38
   wherein, if the expression level of said at least two nucleic acids is above a threshold value, the sample is designated as prostate cancer positive.

The invention further relates to a method for the diagnosis and/or treatment of prostate cancer comprising the steps of analysing the expression level of at least two nucleic acids in a sample of a patient, wherein said at least two nucleic acids are selected from at least two groups of nucleic acids, wherein said groups consist of:
Group 1: SEQ ID NO 1

Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38
   wherein, if the expression level of said at least two nucleic acids is above a threshold value, the sample is designated as prostate cancer positive; and administering to the patient one or more Prostate Cancer Therapeutic Agents.

Preferred Prostate Cancer Therapeutic Agents comprise: Docetaxel (Taxotere®); Cabazitaxel (Jevtana®); Mitoxantrone (Novantrone®); Estramustine (Emcyt®); Doxorubicin (Adriamycin®); Etoposide (VP-16); Vinblastine (Velban®); Paclitaxel (Taxol®); Carboplatin (Paraplatin®); Abiraterone acetate, Bicalutamide, Casodex, Degarelix, Enzalutamide, Goserelin acetate, Leuprolide acetate, Prednisone, Sipuleucel-T, Radium 223 dichloride and/or Vinorelbine (Navelbine®)

In a preferred embodiment, the invention relates to a method for the diagnosis of prostate cancer comprising the steps of analysing the expression levels of the at least two nucleic acids, wherein at least one of said at least two nucleic acids is selected from group 2. SEQ ID NO 4, 5, 6, 7, 8, 9 and 10 and/or group 1. SEQ ID NO 1.

In another embodiment of the invention the expression level of at least 3 nucleic acids is analysed, wherein said nucleic acids are selected from at least 3 different groups. In an alternative embodiment of the invention the expression level of at least 4 nucleic acids is analysed, wherein said nucleic acids are selected from at least 4 different groups. In an alternative embodiment of the invention the expression level of at least 5 nucleic acids is analysed, wherein said nucleic acids are selected from at least 5 different groups. In an alternative embodiment of the invention the expression level of at least 6 nucleic acids is analysed, wherein said nucleic acids are selected from at least 6 different groups. In an alternative embodiment of the invention the expression level of at least 7 nucleic acids is analysed, wherein said nucleic acids are selected from at least 7 different groups. In the most preferred embodiment the expression level of at least one nucleic acid from each group is analysed.

The sample could be selected from the group comprising prostate tissue, biopsy material, lymph nodes, urine, ejaculate, blood, blood serum, blood plasma, circulating tumour cells in blood or lymph, any tissue suspected to contain metastases as well as any source that may contain prostate tumour cells or parts thereof, including vesicles like exosomes, micro vesicles, and others as well as free or protein-bound RNA molecules derived from prostate tumour cells. More preferably, the sample is urine, and most preferably, the sample is urine obtained from a patient after a digital rectal examination (DRE).

Any suitable method for the quantification of nucleic acids may be used to analyse the expression levels of the nucleic acids. In one embodiment the quantification of nucleic acids is performed using a fluorescence based assay. In a preferred embodiment the analysis of the expression level is performed by measuring the fluorescence of a labelled primer, labelled probe or a fluorescent detection agent.

In another embodiment of the invention the quantification is performed using an assay based on nucleic acid amplification. In a preferred embodiment the quantification is performed using PCR or sequencing methods. In a most preferred embodiment the quantification of nucleic acid is performed using qRT-PCR.

In an alternative embodiment the proteins, which are encoded by the nucleic acids or their reverse complements are analysed and quantified.

The invention further relates to a kit for the diagnosis of prostate cancer comprising at least two primers or probes, which hybridize under stringent conditions to at least two nucleic acids, wherein said at least two nucleic acids are selected from at least two groups of nucleic acids, wherein said groups consist of
Group 1: SEQ ID NO 1
Group 2: a splice variant of Ensembl gene ID ENSG00000245750.3 selected from the group consisting of SEQ ID NOs 4, 5, 6, 7, 8, 9 and 10
Group 3: a splice variant of Ensembl gene ID ENSG00000255545.3 selected from the group SEQ ID NOs 26, 27, 28 and 29
Group 4: SEQ ID NOs 3 and 11
Group 5: SEQ ID NO 12
Group 6: SEQ ID NO 24
Group 7: SEQ ID NO 36
Group 8: SEQ ID NO 38

The kit may contain more than two nucleic acids. In a preferred embodiment the kit also contains reagents for performing the analysis of the expression level. In a preferred embodiment the kit additionally comprises reagents for nucleic acid amplification and/or nucleic acid quantification and/or nucleic acid detection. In another embodiment the kit comprises control samples.

The experimental results demonstrate high specificity and sensitivity of the novel biomarker combinations for the detection of PCa.

Ideally, the expression levels of transcripts of the nucleic acids of the groups are compared to the expression level of transcript of one or several other genes in the sample, such as of housekeeping genes. Examples of suitable housekeeping genes are shown below in Table 2:

TABLE 2

| | Housekeeping name |
|---|---|
| Housekeeping | GAPDH—Glyceraldehyde 3-phosphate dehydrogenase |
| | HPRT1—hypoxanthine phosphoribosyltransferase 1 |
| | HMBS—hydroxymethylbilane synthase |
| | TBP Tata box binding protein |

The threshold value is the minimal expression difference between the test sample and the control sample at which the sample is designated as cancer-positive.

Ideally the expression level difference of the biomarker between the test sample and the control sample is 1.5 fold (±20%), 2 fold (±20%), 3 fold (±20%), 4 fold (±20%) and most preferably 5-fold (±20%) or more. The p-value (T test) is <2×10⁻⁵. The FDR is preferably <5×10−4.

For the selected groups of genes, the threshold value is preferably a 2 fold (±20%) expression increase between tumour and control sample.

The invention relates to the analysis of RNA biomarker levels. This can be accomplished by a number of methods, for instance PCR-based methods like quantitative reverse transcription PCR.

The invention relates to a method of amplification to specifically determine the level of biomarkers. Herein, the sample is mixed with forward and reverse primers that are specific for nucleic acids of at least sequences selected from the following groups of sequences:
Group 1: SEQ ID NO 1
Group 2: a splice variant of Ensembl gene ID ENSG00000245750.3 selected from the group consisting of SEQ ID NO: 4, 5, 6, 7, 8, 9 and 10
Group 3: a splice variant of Ensembl gene ID ENSG00000255545.3 selected from the group SEQ ID NO: 26, 27, 28 and 29
Group 4: SEQ ID NO 3 and 11
Group 5: SEQ ID NO 12
Group 6: SEQ ID NO 24
Group 7: SEQ ID NO 36
Group 8: SEQ ID NO 38
   and amplification cycles are performed. Probes or primers are designed such that they hybridize under stringent conditions to said target sequence. The at least two amplification reactions may be performed in parallel or in form of a multiplex amplification.

In one embodiment, the analysis in the method is performed by measuring the fluorescence of a labelled primer, labelled probe or a fluorescent detection agent. More preferably, the analysis of the expression level is performed by qRT-PCR.

In a further embodiment the analysis of the expression level of the at least two nucleic acids is performed using a sequencing method.

The invention also relates to a quantification of the expression levels of the biomarkers to included in the combinations. After amplification, quantification is straightforward and can be accomplished by a number of methods. In the case when primers are used wherein at least one primer has a fluorescent dye attached, quantification is possible using the fluorescent signal from the dye. Various primer systems and dyes are available, such as SYBR green, Multiplex probes, TaqMan probes, molecular beacons and Scorpion primers. Other possible means of quantification are for example northern blotting, next generation sequencing or absorbance measurements at 260 and 280 nm.

As will become clear from the examples below, the invention discloses biomarker combinations for prostate cancer, which allow a more accurate and sensitive diagnosis of the disease than current biomarkers.

### EXAMPLES

### Clinical Cohort

Prostate carcinoma (PCa) patients who underwent radical prostatectomy (RPE) or surgery to remove a benign prostate hyperplasia (BPH) at the University Hospital of Dresden were included in a retrospective clinical cohort aiming at identifying novel biomarkers for PCa. Approval from the local ethics committee as well as informed consent from the patients were obtained according to the legal regulations. For PCa patients, data on the clinical follow-up for at least five years were collected.

Prostate tissue samples from a cohort of 40 PCa patients and 8 BPH patients were used for identification of diagnostically relevant biomarkers by genome-wide RNA sequencing..

Four PCa groups were defined based on staging according to Gleason (The Veteran's Administration Cooperative Urologic Research Group: histologic grading and clinical staging of prostatic carcinoma; in Tannenbaum, M. Urologic Pathology: The Prostate, Philadelphia: Lea and Febiger. Pp. 171-198) as well as the presence of metastases in the adjacent lymph nodes upon RPE (see Table 3).

TABLE 3
PCa cohort for genome-wide RNA sequencing: The control group (C) consists of
BPH samples. The very low risk (V) and low risk (L) groups compris samples from
patients graded with Gleason Score (GS) <7 and =7, respectively, all without lymph node
metastases (pN0). The medium risk (M) group comprise cases with GS <=7 and exhibiting
lymph node metastases (pN+); and the high risk (H) group consist of tissues with GS >7.

| | Group | | | | | | | |
|---|---|---|---|---|---|---|---|---|
| | C | V | L | M | H | | | |
| Gleason score | BPH | GS <7 | GS =7 | GS <=7 | GS >7 | | | |
| lymph node metastasis | — | pN0 | pN0 | pN+ | pN0 | | pN+ | |
| tissue | control | tumour | tumour | tumour | tumour | tumour- | tumour | tumour- |
| | | | | | | free | | free |
| number of samples | 8 | 8 | 8 | 8 | 8 | 8 | 8 | 8 |

For the latter, pairs of tumour and tumour-free tissue samples obtained from the same patient were analysed.

Selected biomarker candidates were further validated by custom microarrays and quantitative reverse-transcription real-time PCR (qRT-PCR) on cohorts comprising 256 (40 control BPH, 216 tumour samples) and 56 patients (16 control BPH samples, 40 tumour samples), respectively.

### Prostate Tissue Samples

Prostate tissue samples were obtained from surgery carried out at the Dept. of Urology of the University Hospital of Dresden and stored in liquid nitrogen at the Comprehensive Cancer Centre of Dresden University. Prostate tissue samples obtained from radical prostatectomies (RPEs) of prostate carcinoma (PCa) patients were divided into tumour and tumour-free samples. Prostate tissue samples from patients with benign prostate hyperplasia (BPH) were used as non-tumour controls. Patient consent was always given.

To verify the status of the samples and their tumour cell content, all samples were divided into series of cryosections. To this end, frozen tissue samples were embedded in Tissue-Tek OCT-compound (Sakura Finetek GmbH) and fixed on metal indenters by freezing. Cryosections were prepared using a cryomicrotome (Leica) equipped with a microtome blade C35 (FEATHER) cooled to −28° C. Every sample was cut into a total of 208 cryosections, 4 of which were HE-stained and evaluated by a pathologist with respect to their tumour cell content (FIG. 1). This yielded 3 stacks of consecutive cryosections, each of which was flanked by HE-stained sections. Only stacks that were flanked on either side by sections containing at least 60% or at most 5% tumour cells were used as tumour or tumour-free samples, respectively. 50 cryosections of the stacks chosen were then subjected to RNA preparation.

### RNA Isolation

Total RNA was isolated from cryo-preserved tissue using Qiazol and the miRNeasy Mini Kit on the QIAcube (all from Qiagen) with manual subsequent DNase I digestion. RNA concentration was determined using a Nanodrop 1000 (Peqlab). RNA integrity was verified on an Agilent Bioanalyzer 2100 (Agilent Technologies, Palo Alto, Calif.), and only RNA samples with an RNA-Integrity-Number (RIN) of at least 6 were further processed.

### Genome-Wide Long-RNA Next Generation Sequencing

Genome-wide long RNA sequencing was performed using a subset of the retrospective PCa cohort comprising 8 prostate tissue samples from benign prostate hyperplasia (BPH) as a control and 56 tumour tissue samples (including tumour and tumour free tissue pairs from samples with Gleason score >7). 1 μg of total RNA was depleted of ribosomal RNA using the Ribo-Zero rRNA Removal Kit (Epicentre). Sequencing libraries were prepared from 50 ng of rRNA-depleted RNA using ScriptSeq v2 RNA-Seq. Library Preparation Kit (Epicentre). The di-tagged cDNA was purified using the Agencourt AMPure XP System Kit (Beckman Coulter). PCR was carried out through 10 cycles to incorporate index barcodes for sample multiplexing and amplify the cDNA libraries. The quality and concentration of the amplified libraries were determined using a DNA High Sensitivity Kit on an Agilent Bioanalyzer (Agilent Technologies). 4 ng each of 8 samples were pooled and size-selected on 2% agarose gels using agarose gel electrophoresis. The sample range between 150 bp and 600 bp was gel-excised and purified with the MinElute Gel Extraction Kit (Qiagen), according to manufacturer's instructions. The purified libraries were quantified on an Agilent Bioanalyzer using a DNA High Sensitivity Chip (Agilent Technologies). Every purified and size-selected library pool was then loaded onto an Illumina HiSeq2000 flow cell, distributing it among all lanes. Cluster generation was performed using TruSeq PE Cluster Kits v3 (Illumina Inc.) in an Illumina cBOT instrument following the manufacturer's protocol. Sequencing was performed on an Illumina HiSeq2000 sequencing machine (Illumina, Inc.). The details of the sequencing runs were as follows: paired-end sequencing strategy; 101 cycles for Read1, 7 cycles for index sequences, and 101 cycles for Read2.

### Analysis of Sequencing Data: Raw Data Preparation

Raw sequencing data comprising base call files (BCL files) was processed with CASAVA v1.8.1 (Illumina) resulting in FASTQ files. FASTQ files contain for each clinical sample all sequenced RNA fragments, in the following referred to as “reads”. Specific adapter sequences were removed by using cutadapt (http://code.google.com/p/cutadapt/).

### Analysis of Sequencing Data: Genome Mapping and Transcript Assembling:

Reads were mapped to the human genome (assembly hg19) using segemehl v0.1.4-382 and TopHat v2.0.9. Novel transcripts, i.e. transcripts not annotated in Gencode v17, were assembled using Cufflinks v2.1.1 and Cuffmerge v2.1.1, All novel transcripts and all known Gencode v17 transcripts were combined into a comprehensive annotation set.

### Analysis of Sequencing Data: Statistical Analysis

Htseq-count v0.5.4p1 (http://www-huber.embl.de/users/anders/HTSeq/doc/count.html) was used to compute the read counts per transcript and gene that are contained in the comprehensive annotation set of novel and known transcripts. Differentially expressed transcripts and genes were identified using R and the Bioconductor libraries edgeR. Different RNA composition of the clinical samples was adjusted for by scaling library size for each sample (TMM method). A negative binomial log-linear model was fitted to the read counts for each transcript or gene, and coefficients distinct from zero identified by a likelihood ratio test. False discovery rate was controlled by Benjanimi-Hochberg adjustment.

### Validation By Custom Microarrays

Based on the sequencing results custom microarrays with 180 000 probes (Agilent SurePrint G3 Custom Exon Array, 4×180K, Design-ID 058029) were designed comprising mRNAs, long non coding RNAs (gencode v15), new transcripts and all differential expressed transcripts.

The microarray screening was performed using the retrospective PCa cohort comprising 40 prostate tissue samples from benign prostate hyperplasia as a control as a control, as well as 164 and 52 tumour and tumour-free tissue samples, respectively, of PCa patients after radical prostatectomy. Using the Quick Amp Labeling Kit (Agilent) cRNA was synthesized from 200 ng total RNA, and 1650 ng cRNA was hybridized on the arrays (Agilent Gene Expression Hybridization Kit).

### Validation By Quantitative Real-Time PCR

For validation of the results obtained by next generation sequencing and microarray screening a total of 56 tissue samples (16 tumour free and 40 tumor samples) were screened using quantitative real-time PCR.

cDNA was synthesized from 100 ng total RNA using the High-Capacity Reverse transcription kit (Applied Biosystems) and random primers according to manufacturer's instructions. Subsequent PCR assays were run using 4 μl of the diluted cDNA. Quantitative real-time PCR was performed using custom- and pre-designed TaqMan Gene Expression Assays (Applied Biosystems) for housekeeping and target transcripts on an Applied Biosystems 7900HT Real-Time PCR System.

All samples were measured in triplicate and the means of these measurements were used for further calculations.

TABLE 4
IDs of the Applied Biosystems TaqMan Gene Expression
Assays used for qRT-PCR validation in prostate tissue samples.

| | Housekeeping/Target name | TaqMan Assay ID |
|---|---|---|
| Housekeeping | GAPDH | Hs02758991_g1 |
| | HPRT1 | Hs02800695_m1 |
| | HMBS | Hs00609293_g1 |
| Target | SEQ ID NO 1 | AJ70L28 |
| | SEQ ID NO 9 | Hs01388451_m1 |
| | SEQ ID NO 3 | AJCSVRJ |
| | PCA3 | Hs01371939_g1 |

### Statistical Analysis of the RT-qPCR Results

Data normalization was carried out against the unregulated housekeeping genes GAPDH (SEQ ID NO 1) and HPRT1 (SEQ ID NO 3 and SEQ ID NO 9), respectively. For relative quantification, changes in gene expression of each sample were analysed relative to the median expression of the control samples. All statistical analyses were carried out using R statistical software.

The log2-transformed relative expression levels of the biomarkers were compared between tumor and control samples employing Student's t-test. Receiver-operating characteristic (ROC) curves, representing a measure of diagnostic power of each marker by the area under the curve (AUC), were calculated using the package pROC.

All samples were measured in triplicate and the means of these measurements were used for further calculations.

### Validation in DRE Urine Samples

### DRE Urine Sample Collection and RNA Isolation

Urine samples were collected after digital rectal examination (DRE) of the prostate (DRE urine). This routinely performed examination method allows getting urine samples including a certain amount of prostate cells. The DRE urine samples were centrifuged and washed two times using PBS. The resulting cell pellet was resuspended in 700 μl Qiazol. Total RNA was isolated using the miRNeasy Mini Kit on the QlAcube (all from Qiagen) with manual subsequent DNase I digestion. RNA concentration was determined using a Nanodrop 1000 (Peqlab). RNA integrity was verified on an Agilent Bioanalyzer 2100 (Agilent Technologies, Palo Alto, Calif.).

Quantitative Real-Time PCR Screening of DRE Urine Samples cDNA was synthesized from 2×50 ng total RNA using the Superscript III Reverse transcriptase (Applied Biosystems) and random primers according to manufacturer's instructions. Subsequent PCR assays were run using 4 μl of cDNA. Quantitative real-time PCR was performed using custom and pre-designed TaqMan Gene Expression Assays (Applied Biosystems) for housekeeping (PSA) and target transcripts on an Applied Biosystems 7900HT Real-Time PCR System.

All samples were measured in duplicate and the means of these measurements were used for further calculations.

### Genome-Wide Long-RNA Next Generation Sequencing of DRE Urine Samples

For genome-wide long RNA sequencing total RNA from 7 DRE urine samples was precipitated using ethanol to concentrate the RNA amount and resuspended in 10 μl RNase free water. The rRNA removal was performed with 4 ng of total RNA using the Low input

Ribo-Zero rRNA Removal Kit (Epicentre modified by clontech), resulting in 10 μl rRNA depleted RNA. Sequencing libraries were prepared from 8μ1 rRNA-depleted RNA using the SMARTER stranded RNAseq Kit (Clontech). The di-tagged cDNA was purified using the Agencourt AMPure XP System Kit (Beckman Coulter). PCR was carried out through 18 cycles to incorporate index barcodes for sample multiplexing and amplify the cDNA libraries. The quality and concentration of the amplified libraries were determined using a DNA High Sensitivity Kit on an Agilent Bioanalyzer (Agilent Technologies). Samples were pooled and cluster generation was performed using 15 pmol/l of the pooled library and the TruSeq PE Cluster Kit v4 (Illumina Inc.) in an Illumina CBOT instrument following the manufacturer's protocol. Sequencing was performed using the HiSeq SBS v4 sequencing reagents (250 cycles) on an Illumina HiSeq2500 sequencing machine (Illumina, Inc.). The details of the sequencing run were as follows: paired-end sequencing strategy; 126 cycles for Read1, 7 cycles for index sequences, and 126 cycles for Read2.

Statistical Analysis of the qRT-PCR Results from DRE Urines

For analysis of qRT-PCR results from DRE urine samples data normalization was carried out against the prostate specific antigen (PSA). For relative quantification, changes in gene expression of each sample were analysed relative to the median expression of the control samples. All statistical analyses were carried out using R statistical software.

TABLE 5
IDs of the Applied Biosystems TaqMan Gene Expression
Assays used for qRT-PCR validation.

| | Name | TaqMan Assay ID |
|---|---|---|
| Housekeeping | GAPDH | Hs02758991_g1 |
| | HPRT1 | Hs02800695_m1 |
| Target | SeqID 1 | AJ70L28 |
| | PSA | Hs02576345_m1 |

### Results

The transcriptomes of 40 PCa tumour samples and 16 tumour-free samples obtained upon

RPE and 8 BPH prostate tissue samples as benign, non-tumour controls were analysed using strand-specific, paired-end long RNA next generation sequencing (NGS). Approximately 150 cryosections per sample in at least three segments were prepared, aiming at an optimal data quality and robustness of the analysis. Upon pathological evaluation, only segments satisfying a maximal and minimal tumour cell count of 60% and 5% in tumour and tumour free samples, respectively, were retained for further analysis. The transcriptome sequencing (RNAseq) approach aimed at a comprehensive identification and quantification of RNAs expressed in normal or cancer prostate tissue. All classes of coding and long non-coding transcripts independent of polyadenylation status were sequenced. Large input masses of RNA were used to ensure high library complexity. Furthermore, on average 200 M paired-end reads 2×100 nt per library were sequenced, enabling the assembly of novel lowly expressed transcripts due to high coverage. This approach outperformed most comparable published studies that analyzed larger numbers of samples. In total, approx. 3000 novel transcripts that did not show an exonic overlap with transcripts annotated in Gencode v17 were assembled. At a false discovery rate of 0.01, 6442 differentially expressed genes across all contrasts were observed. Numbers of differentially expressed genes for specific contrasts are given in Table 4.

TABLE 6
Number of differentially expressed genes for diverse contrasts and Gencode
biotypes.

| | | Protein | | | Sense- | | Novel | Non-protein |
|---|---|---|---|---|---|---|---|---|
| Contrast | Total | coding | lincRNA | Antisense | intronic | Pseudogene | transcript | coding |
| Tumour vs. Control | 5615 | 3882 | 116 | 96 | 13 | 456 | 847 | 1733 |
| Tumour Gleason >7 vs. | 2677 | 1812 | 73 | 40 | 4 | 88 | 552 | 865 |
| control | | | | | | | | |
| Tumour high and | 138 | 51 | 3 | 2 | 0 | 7 | 72 | 87 |
| medium vs. Tumour low | | | | | | | | |
| and very low | | | | | | | | |
| Tumour Gleason = 7 vs. | 12 | 6 | 0 | 1 | 0 | 0 | 5 | 6 |
| Tumour Gleason <7 | | | | | | | | |
| Tumour Gleason >7 vs. | 14 | 7 | 0 | 0 | 0 | 1 | 6 | 7 |
| Tumour Gleason = 7 | | | | | | | | |

The results successfully reproduced the majority of transcripts previously reported to be differentially expressed between prostate tumour and normal tissue. In addition, a number of novel PCa-associated transcripts were identified, which can be used to develop assays for the diagnosis of PCa. Selected most promising transcripts were validated in a test cohort of PCA tumour and BPH control samples by qRT-PCR.

Several of these novel biomarker candidates significantly surpass the specificity and sensitivity of the biomarker PCA3, which is already used for PCa diagnosis. In the sequencing cohort, PCA3 proved to be clearly associated with PCa, yet with a strong tendency to a decline in the high-risk group (FIG. 2).

In the test cohort, the novel biomarker Retro-RPL7 (SEQ ID NO 1) yielded an area under ROC curve (AUC) value of 0.935, compared to 0.851 for PCA3 (FIG. 3). A combination of our novel biomarkers Retro-RPL7 and XLOC133897 showed an AUC of 0.975 (FIG. 6).

The experimental results demonstrate high specificity and sensitivity of the novel biomarkers for the detection of PCa. Therefore, assays can be set up based on the measurement of these newly discovered biomarkers alone or in combination (or in combination with already known markers) in all sources that may contain prostate tumour cells or parts thereof (including vesicles like exosomes, microvesicles, and others as well as free or protein-bound RNA molecules deriving from prostate tumour cells) to be used for the diagnosis of PCa. These sources include (but are not limited to) prostate tissue, biopsy material, lymph nodes, urine, ejaculate, blood, blood serum, blood plasma, circulating tumour cells in blood or lymph, as well as any tissue suspected to contain PCa metastases. Measurement of our RNA biomarkers can be done by any method suited to specifically estimate RNA levels, e.g. PCR-based methods like qRT-PCR. The assays can be applied for early diagnosis (screening) of PCa, for predicting the aggressiveness of the tumours (prognosis), and/or for aiding the choice of therapy.

Diagnostic assays based on these biomarkers may therefore dramatically decrease the high false-positive rates of current assays and thereby help to avoid unnecessary invasive prostate biopsies.

Combinations of biomarkers selected from the groups consisting of:
Group 1: SEQ ID NO 1
Group 2: a splice variant of Ensembl gene ID ENSG00000245750.3 selected from the group consisting of SEQ ID NO: 4, 5, 6, 7, 8, 9 and 10
Group 3: a splice variant of Ensembl gene ID ENSG00000255545.3 selected from the group SEQ ID NO: 26, 27, 28 and 29
Group 4: SEQ ID NO 3 and 11
Group 5: SEQ ID NO 12
Group 6: SEQ ID NO 24
Group 7: SEQ ID NO 36
Group 8: SEQ ID NO 38
   have also been analysed for their diagnostic value in combination. The inventors found that the diagnostic value and the AUC greatly increased, when a combination of said groups was analyzed as shown in the following tables and FIG. 7. This indicates that not only are the single novel biomarkers superior to previously known biomarkers, but that their combination surprisingly allows further substantial improvement of the PCa diagnosis.

TABLE 7
AUC values of marker combinations involving sequences from group
5, compared to the AUC value of group 5 alone. Data derive from
custom microarray analysis of 256 prostate tissue samples.

| Seq A | | Seq B | AUC value |
|---|---|---|---|
| SeqID 12 | | | 0.817 |
| SeqID 12 | + | SeqID 1 | 0.985 |
| SeqID 12 | + | SeqID 3 | 0.959 |
| SeqID 12 | + | SeqID 11 | 0.96 |
| SeqID 12 | + | SeqID 4_10 | 0.989 |
| SeqID 12 | + | SeqID 38 | 0.896 |
| SeqID 12 | + | SeqID 36 | 0.944 |

TABLE 8
AUC values of marker combinations involving sequences from
group 2, compared to the AUC value of group 2 alone. Data derive
from custom microarray analysis of 256 prostate tissue samples

| Seq A | | Seq B | AUC array |
|---|---|---|---|
| SeqID 4_10 | | | 0.959 |
| SeqID 4_10 | + | SeqID 26_29 | 0.981 |
| SeqID 4_10 | + | SeqID 12 | 0.989 |
| SeqID 4_10 | + | SeqID 38 | 0.98 |
| SeqID 4_10 | + | SeqID 36 | 0.988 |

TABLE 9
AUC values of marker combinations involving sequences from
group 3, compared to the AUC value of group 3 alone. Data derive
from custom microarray analysis of 256 prostate tissue samples.

| Seq A | | Seq B | AUC array |
|---|---|---|---|
| SeqID 26_29 | | | 0.849 |
| SeqID 26_29 | + | SeqID 12 | 0.926 |
| SeqID 26_29 | + | SeqID 38 | 0.915 |
| SeqID 26_29 | + | SeqID 36 | 0.954 |
| SeqID 26_29 | + | SeqID 4_10 | 0.981 |
| SeqID 26_29 | + | SeqID 1 | 0.985 |
| SeqID 26_29 | + | SeqID 3 | 0.962 |
| SeqID 26_29 | + | SeqID 11 | 0.963 |

TABLE 10
AUC values of marker combinations involving sequences from group
4, compared to the AUC value of group 4 alone. Data derive from
custom microarray analysis of 256 prostate tissue samples

| Seq A | | Seq B | AUC array |
|---|---|---|---|
| SeqID 3 | | | 0.935 |
| SeqID 3 | + | SeqID 1 | 0.984 |
| SeqID 3 | + | SeqID 4_10 | 0.981 |
| SeqID 3 | + | SeqID 11 | 0.946 |
| SeqID 3 | + | SeqID 12 | 0.959 |
| SeqID 3 | + | SeqID 26_29 | 0.962 |
| SeqID 3 | + | SeqID 36 | 0.974 |
| SeqID 3 | + | SeqID 38 | 0.958 |

TABLE 11
AUC values of marker combinations involving sequences from
group 1, compared to the AUC value of group 1 alone. Data derive
from custom microarray analysis of 256 prostate tissue samples

| Seq A | | Seq B | AUC array |
|---|---|---|---|
| SeqID 1 | | | 0.945 |
| SeqID 1 | + | SeqID 3 | 0.984 |
| SeqID 1 | + | SeqID 4_10 | 0.986 |
| SeqID 1 | + | SeqID 11 | 0.983 |
| SeqID 1 | + | SeqID 12 | 0.985 |
| SeqID 1 | + | SeqID 26_29 | 0.985 |
| SeqID 1 | + | SeqID 36 | 0.986 |
| SeqID 1 | + | SeqID 38 | 0.983 |

TABLE 12
AUC values of marker combinations involving sequences
from group 6, compared to the AUC value of group 6 alone.
Data derive from RNA next-generation sequencing
analysis of 64 prostate tissue samples.

| Seq A | | Seq B | AUC sequencing |
|---|---|---|---|
| SeqID 24 | | | 0.919 |
| SeqID 24 | + | SeqID 1 | 1 |
| SeqID 24 | + | SeqID 3 | 0.984 |
| SeqID 24 | + | SeqID 11 | 0.975 |
| SeqID 24 | + | SeqID 12 | 1 |
| SeqID 24 | + | SeqID 4_10 | 0.988 |
| SeqID 24 | + | SeqID 26_29 | 0.962 |
| SeqID 24 | + | SeqID 36 | 0.959 |
| SeqID 24 | + | SeqID 38 | 0.966 |

TABLE 13
AUC values of marker combinations involving sequences from group
7 and 8, compared to the AUC values of group 7 or 8 alone. Data
derive from custom microarray analysis of 256 prostate tissue samples.

| Seq A | | Seq B | AUC value |
|---|---|---|---|
| SeqID 38 | | | 0.806 |
| | | SeqID 36 | 0.88 |
| SeqID 38 | + | SeqID 36 | 0.958 |

The inventors also found that the expression of all groups could be detected in urine sample of patients, while being absent or low expressed in healthy patients (FIG. 8). This is surprising because Fontenete et al., (Int. braz j urol. vol. 37 no. 6 Rio de Janeiro November/December 2011) showed that the mRNA of PSA is not a suitable biomarker for prostate cancer in urine samples, as it was found to be overexpressed more frequently in healthy patients than in PCa patients in these samples. Therefore, it was not a priori evident that analysing the biomarker expression levels in urine samples could be used to reliably diagnose prostate cancer.

The advantages of a diagnostic assays based on these biomarker combinations allows a dramatically lower false-positive rate compared to current assays and measuring their expression levels in urine sample avoid having to perform unnecessary invasive prostate biopsies.

### FIGURE CAPTIONS

FIG. 1: Verification of tissue sample quality: to determine the tumour cell content of the tissue samples, cryosections were prepared from the frozen samples as shown. HE: hematoxylin/eosin; IHC: immunohistochemistry. Verification of tissue sample quality: cryosections of 4 μm were prepared from the frozen samples as shown for HE staining (to ensure tumour cell content of the tissue samples), for RNA and DNA isolation and for IHC. HE: hematoxylin/eosin; IHC: immunohistochemistry.

FIG. 2: Box-blot of RNA-seq data for transcript PCA3. Results from RNA sequencing of the retrospective PCa cohort comprising 8 prostate tissue samples from benign prostate hyperplasia as a control (C), 8 PCa tumour samples each of groups V (very low risk; Gleason<7, pN0), L (low risk; Gleason score=7, pN0), and M (medium risk; Gleason score<=7, pN+), as well as 16 pairs of tumour and tumour-free tissue samples from group H (high risk; Gleason score >7).

FIG. 3: ROC curves of Retro-RPL7 (SEQ ID NO 1) and PCA3 resulting from qRT-PCR analysis of 56 prostate tissue samples.

FIG. 4: Box-blot of custom microarray data for SEQ ID NO 12. Results from custom microarray analysis of the retrospective PCa cohort comprising 40 prostate tissue samples from benign prostate hyperplasia (BPH) as a control, as well as 164 and 52 tumour and tumour-free tissue samples, respectively, of PCa patients after radical prostatectomy (RPE).

FIG. 5: ROC curve of SEQ ID NO 12 resulting from custom microarray analysis of 256 prostate tissue samples as described in FIG.4.

FIG. 6: Logit model based on a two-transcript signature. ROC curve is shown for the multivariate logit model comprising Retro-RPL7 (SEQ ID NO 1) and XLOC133897 (SEQ ID NO 2) (AUC=0.975).

FIG. 7: Biomarker signature: A) Data obtained by RNA next-generation sequencing from 8 control tissue samples (benign prostate hyperplasia, BPH) and 40 prostate carcinoma tissue samples for all transcripts of SEQ ID NOs 1, 3, 4-10, 11, 12, 24, 26-29, 36, and 38 were combined as a signature to yield higher specificity and sensitivity. Data are shown by box plot (left) and ROC curve (right). The resulting AUC value is 1.0. B) Data obtained by custom microarray analysis from 40 control tissue samples (benign prostate hyperplasia, BPH) and 164 prostate carcinoma tissue samples for all transcripts of SEQ ID NOs 1, 3, 4-10, 11, 12, 24, 26-29, and 38 were combined as a signature to yield higher specificity and sensitivity. Data are shown by box plot (left) and ROC curve (right). The resulting AUC value is 1.0.

FIG. 8: Biomarker detection in urine samples by RNA next-generation sequencing: Urine samples were obtained after digital rectal examination of patients by an urologist. RNA isolated from these samples was subjected to transcriptome-wide RNA sequencing using an Illumina HiSeq2500 next-generation sequencer. Reads were mapped to the genome by standard algorithms, and reads mapping to the genomic loci of the SEQ ID NOs shown were counted and normalized to reads derived from the gene locus of prostate-specific antigen (PSA) as a measure for the presence of prostate epithelium cells in the urine. Samples of patients diagnosed with and without prostate cancer were compared.

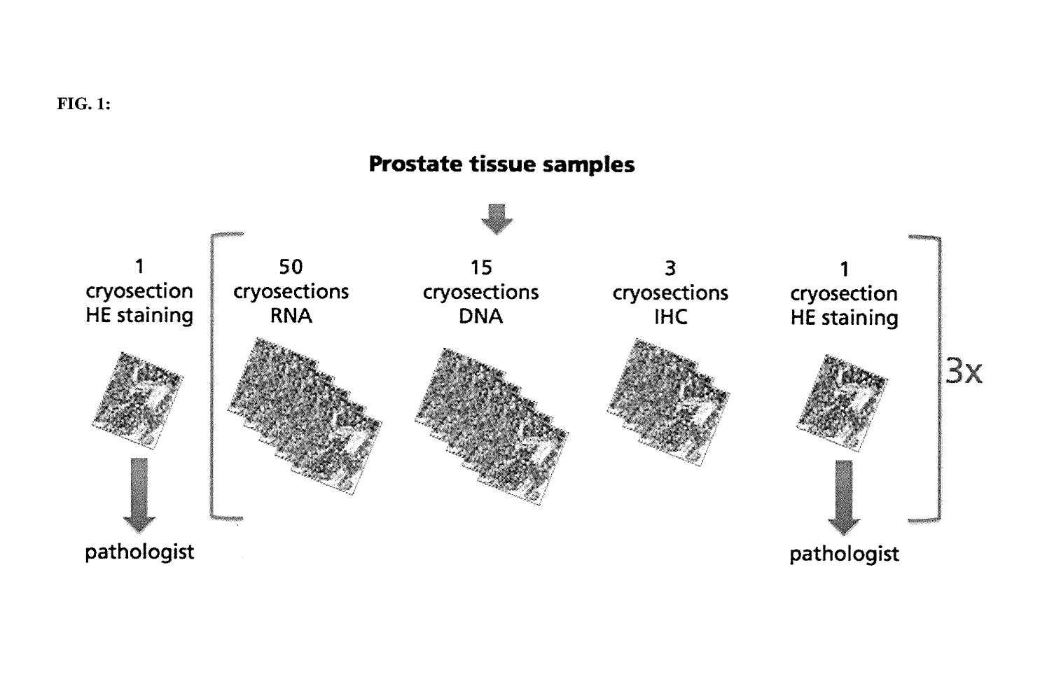

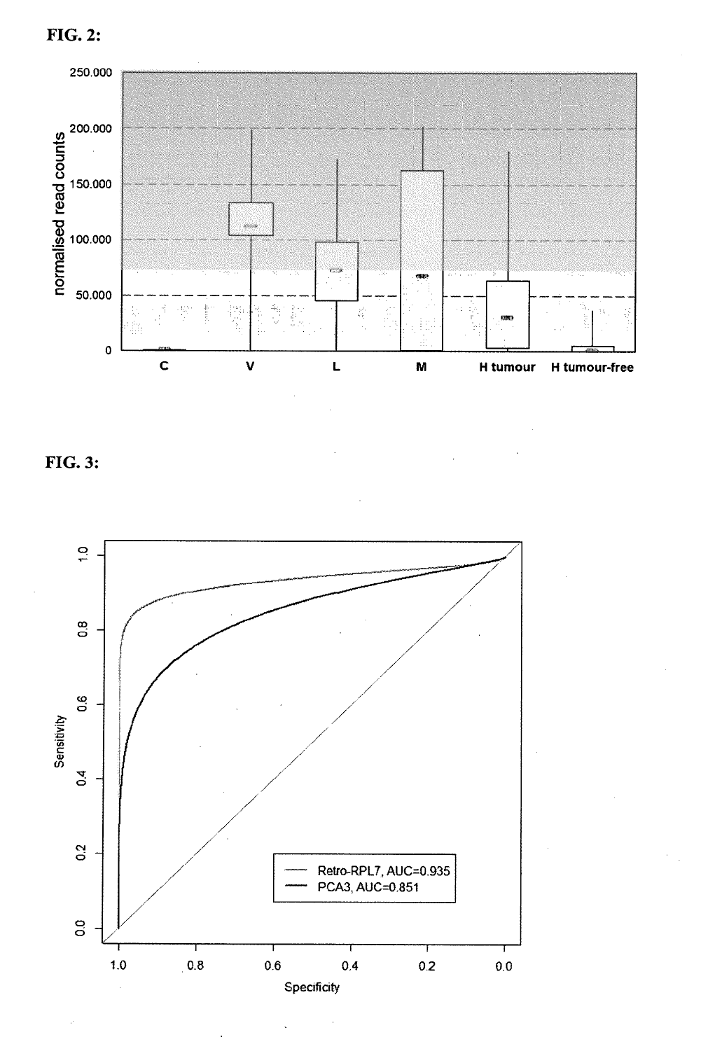

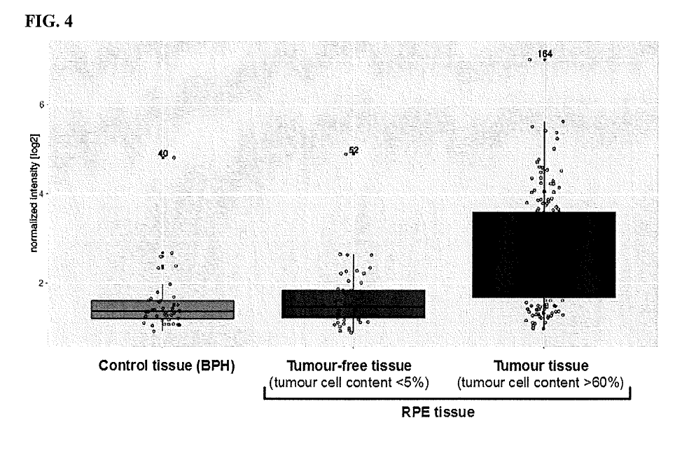

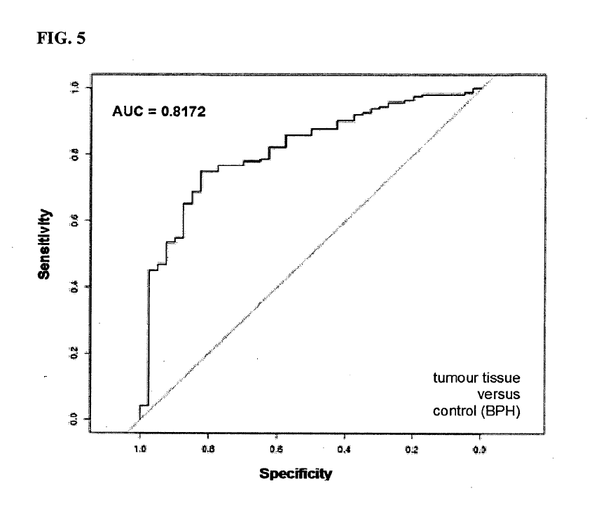

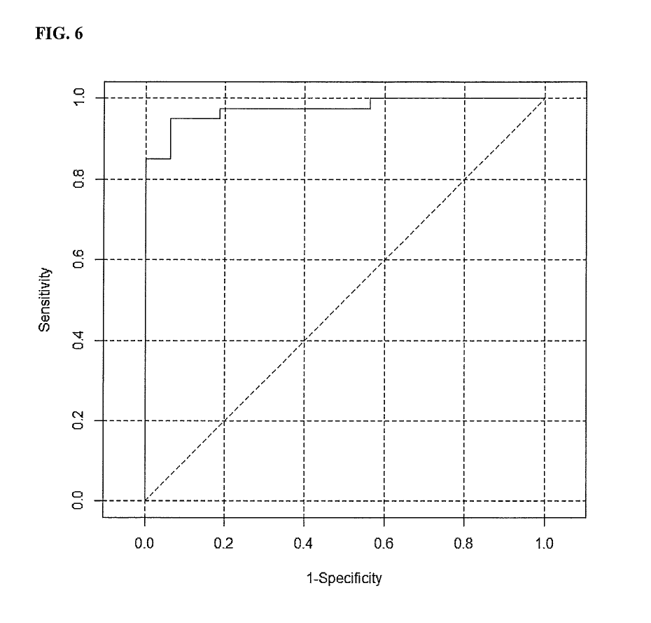

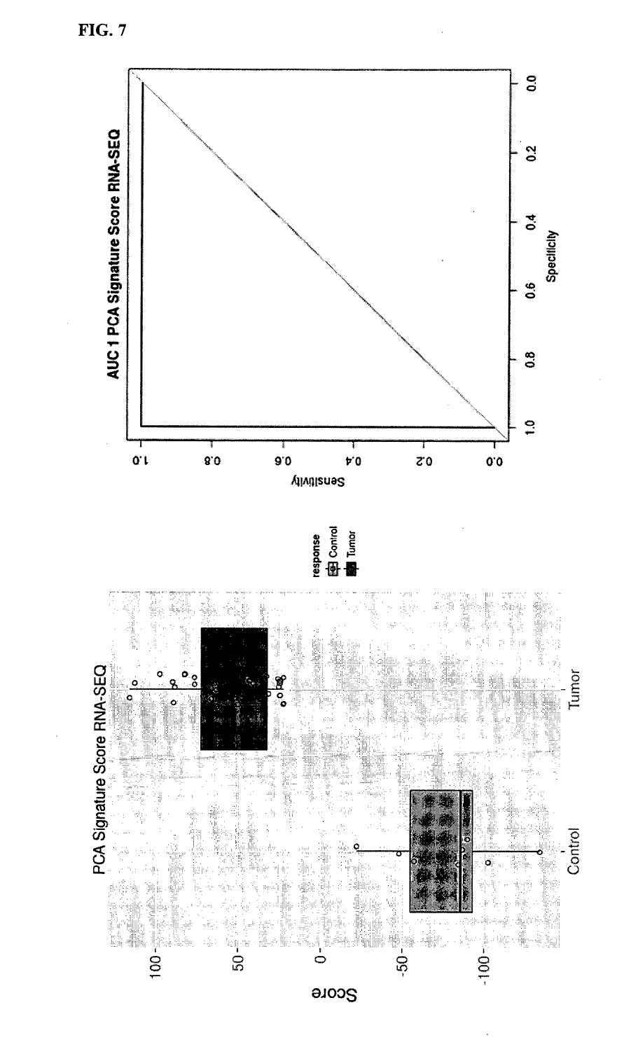

1. A method for the diagnosis of prostate cancer, comprising the steps of
a) analysing the expression levels of at least two nucleic acids in a sample of a patient, wherein said at least two nucleic acids are selected from at least two groups of nucleic acids, wherein said groups consist of:
Group 1: SEQ ID NO 1
Group 2: a splice variant of Ensembl gene ID ENSG00000245750.3 selected from the group consisting of SEQ ID NO: 4, 5, 6, 7, 8, 9 and 10
Group 3: a splice variant of Ensembl gene ID ENSG00000255545.3 selected from the group SEQ ID NO: 26, 27, 28 and 29
Group 4: SEQ ID NO 3 and 11
Group 5: SEQ ID NO 12
Group 6: SEQ ID NO 24
Group 7: SEQ ID NO 36
Group 8: SEQ ID NO 38
b) wherein, if the expression level of said at least two nucleic acids is above a defined threshold value, the sample is designated as prostate cancer positive.

2. A method according to claim 1, wherein at least one of said at least two nucleic acids is selected from group 1 SEQ ID NO 1 and/or group 2 SEQ ID NO 4, 5, 6, 7, 8, 9 and 10.

3. A method according to claim 1 or 2, wherein the expression level of at least 5 nucleic acids are analysed, wherein said nucleic acids are selected from at least 5 different groups.

4. A method according to claim 1, wherein the expression level of at least one nucleic acid from each of the eight groups is analyzed.

5. A method according to any of claims 1 to 4, wherein the sample is selected from the group comprising prostate tissue, biopsy material, lymph nodes, urine, ejaculate, blood, blood serum, blood plasma, circulating tumour cells in blood or lymph, any tissue suspected to contain metastases as well as any source that may contain prostate tumour cells or parts thereof, including vesicles like exosomes, micro vesicles, and others as well as free or protein-bound RNA molecules derived from prostate tumour cells.

6. A method according to any of claims 1 to 4, wherein the sample is a urine sample.

7. A method according to any of the preceding claims, wherein the analysis of the expression level is performed by measuring the fluorescence of a labelled primer, labelled probe or a fluorescent detection agent.

8. A method according to any of the preceding claims, wherein the analysis of the expression level is performed by qRT-PCR.

9. A kit for the diagnosis of prostate cancer comprising at least two primers or probes, which hybridize under stringent conditions to at least two nucleic acids, wherein said at least two nucleic acids are selected from at least two groups of nucleic acids, wherein said groups consist of:
Group 1: SEQ ID NO 1
Group 2: a splice variant of Ensembl gene ID ENSG00000245750.3 selected from the group consisting of SEQ ID NO: 4, 5, 6, 7, 8, 9 and 10
Group 3: a splice variant of Ensembl gene ID ENSG00000255545.3 selected from the group SEQ ID NO: 26, 27, 28 and 29
Group 4: SEQ ID NO 3 and 11
Group 5: SEQ ID NO 12
Group 6: SEQ ID NO 24
Group 7: SEQ ID NO 36
Group 8: SEQ ID NO 38

10. A kit according to claim 9 comprising at least 8 primers or probes, wherein said at least 8 primers or probes hybridize under stringent conditions to at least one nucleic acid of each of the eight groups.

11. A method for the diagnosis and treatment of prostate cancer, comprising the steps of
a. analysing the expression levels of at least two nucleic acids in a sample of a patient, wherein said at least two nucleic acids are selected from at least two groups of nucleic acids, wherein said groups consist of:
Group 1: SEQ ID NO 1
Group 2: a splice variant of Ensembl gene ID ENSG00000245750.3 selected from the group consisting of SEQ ID NO: 4, 5, 6, 7, 8, 9 and 10
Group 3: a splice variant of Ensembl gene ID ENSG00000255545.3 selected from the group SEQ ID NO: 26, 27, 28 and 29
Group 4: SEQ ID NO 3 and 11
Group 5: SEQ ID NO 12
Group 6: SEQ ID NO 24
Group 7: SEQ ID NO 36
Group 8: SEQ ID NO 38
b. wherein, if the expression level of said at least two nucleic acids is above a defined threshold value, the sample is designated as prostate cancer positive; and
c. administering to the patient one or more Prostate Cancer Therapeutic Agents.

12. The method of claim 11, wherein the Prostate Cancer Therapeutic Agents comprises: Docetaxel (Taxotere®); Cabazitaxel (Jevtana®); Mitoxantrone (Novantrone®); Estramustine (Emcyt®); Doxorubicin (Adriamycin®); Etoposide (VP-16); Vinblastine (Velban®); Paclitaxel (Taxol®); Carboplatin (Paraplatin®); Abiraterone acetate, Bicalutamide, Casodex, Degarelix, Enzalutamide, Goserelin acetate, Leuprolide acetate, Prednisone, Sipuleucel-T, Radium 223 dichloride and/or Vinorelbine (Navelbine®)
